(19) United States
(12) Patent Application Publication
(10) Pub. No.: US 2003/0187412 A1
(43) Pub. Date: Oct. 2, 2003

(54) Odor absorbing device and method

(76) Inventor: David A. Martin, Santa Barbara, CA (US)

Correspondence Address: Michael G. Petit, P.O. Box 91929, Santa Barbara, CA, 93190-1929, US

(21) Appl. No.: 10/339,246

(22) Filed: Jan. 8, 2003

Related U.S. Application Data
(60) Provisional application No. 60/368,148, filed on Mar. 29, 2002.

(51) Int. Cl. A61F 13/15; A61F 13/20

(52) U.S. Cl. 604/359

(57) ABSTRACT

A device for adsorbing odor from human gas using an efficient activated carbon filter supported by a thong, the device being adapted to overcome filter blocking and filter bypass. The device includes a filter portion affixed to a thong in a manner that provides correct positioning of the filter and concealment. The thong employs a surface area contact method operable for removably attaching the thong to an undergarment. The filter is granular activated carbon, preferably coconut shell activated carbon, contained within a porous pouch that is affixed to the thong. The thong is preferably a strap having opposing free ends with adhesive means thereon. The filter is disposed on the strap between the free ends. In operation, the thong is positioned on the body such that the filter overlies the body gas vent. An undergarment is then donned over the thong and the free opposing ends of the thong are folded over the upper edge of the undergarment and releasably attached to the outer surface of the undergarment by the adhesive means.

This application claims the benefit of U.S. Provisional Application No. Serial No. 60/368,148, filed Mar. 29, 2002.

### BACKGROUND OF THE INVENTION

1. Field of the Invention

This invention relates to a device and method for eliminating odor from human gas, or flatulence, and more particularly, to an odor adsorbing thong for unrestrictive adult daily wear.

2. Prior Art

There are now more than 281 million people in the United States. More than one-third of them, almost 97 million people, are now over 45 years old. According to the 2000 census, the median age in the US is the highest it has ever been, reflecting the aging of baby boomers. The most rapid increase was a 49% jump in the 45-54 year old population, fueled mainly by entry into this age group of the first of the baby-boomer generation.

As adults mature, the prevalence of gas, a product of the digestive process, often becomes more frequent. Uncontrollable gas release is becoming a growing problem for older adults, with socially embarrassing consequences. If a very conservative estimate of 5% of Americans over the age of 45 have this problem to the degree that they want to do something about it, that constitutes a group of over 14 million men and women seeking a solution. Unfortunately, there are few marketed products available to manage the problem. Surprisingly, there are no products currently available for removing gas odor that can be worn upon the body. One herbal-tablet product, called: “Bean-O”, claims to reduce gas when sprinkled on foods such as beans, but it proved only marginally effective in testing. Drugs are usually a last resort because of expense and side-effects, but even this option, available with drugs such as GAS-X, is only minimally effective. There are no drugs for daily use to eliminate the odor in gas such as occurs in normally healthy people.

A review of the prior art for removing odor from human gas, reveals that almost all related invention is directed toward abnormal conditions, in which medical devices or large bulky pads might not be objectionable, such as for surgical conditions, medical convalescents, nursing home patients, or incontinent patients. There are few examples of prior art that attempt to eliminate the odor of human gas for fully functional healthy adults, and close examination of the most relevant patents reveals that they do not satisfy fundamental requirements for this type of user.

A review of the prior art begins with numerous surgical and health care inventions with claims directed toward abating objectionable odors from human body fluids or other waste materials. Some only address the abnormal problem of absorbing liquid anal discharge, with no concern for odor, such as Matrullo (U.S. Pat. No. 4,182,335), Flanders (U.S. Pat. No. 2,742,042), and Feeney (U.S. Pat. No. 4,505,707). A few claim or describe the use of activated carbon or charcoal to absorb odor, such as Maggs (U.S. Pat. No. 4,657,808) in a surgical dressing, Juhasz (U.S. Pat. No. 4,715,857) in a wound dressing, and Shuler (U.S. Pat. No. 2,690,415) in an odor absorbent pad for bandages. These devices are designed to absorbing odor from fluid, and will be incapable of filtering odor from an explosive gas stream, which gas will take the path of least resistance and simply by-pass these thick, dense pad assemblies. Campbell (U.S. Pat. No. 4,490,145) with an ostomy pouch and deodorizing filter requires a hose connection to the body for this large bulky device, which is obviously unsuitable for use by normal, active adults, even if it could be adapted to such a use.

There are some inventions concerned with deodorizing normal human liquid waste materials, such as Jackson (U.S. Pat. No. 4,547,195), Goldfarb (U.S. Pat. No. 3,490,454), and Schreiber (U.S. Pat. No. 2,418,907), each with a type of sanitary napkin. It is not obvious how any sanitary napkin could be adapted for removing odor from normal human gas, but if it were attempted, the same result as with the surgical dressings can be expected; the gas will take the path of least resistance and by-pass the dense absorbent sanitary napkin structure.

A relatively small group of patents are, wholly or in part, specifically designed for absorbing odor from human gas. These are Yabrov (U.S. Pat. No. 4,880,417) with a glycerin containing pad, Weimer (U.S. Pat. No. 5,593,398) with plastic coated underwear having a gas filter for abnormal gas; Grosse (U.S. Pat. No. 5,665,081) with a charcoal containing pad, and Conant (U.S. Pat. No. 6,313,371) with a laminated layer of activated charcoal cloth.

Yabrov '417 describes an absorbing, deodorizing, anal pad attached to underwear with adhesive strips or pockets sewn into the underwear. While this invention may be useful in cases of illness or disease, several factors eliminate this product from consideration by healthy adults, for eliminating odor from normal gas in everyday life. Any bulky pad attached to the inside of one's underwear would likely be too uncomfortable for any healthy adult to consider wearing. In addition, the social stigma of having to wear a bulky pad stuffed into the back of one's underwear will be clearly unacceptable to nearly all healthy active adults. Further, an absorbent pad of any type is unnecessary for normally functioning adults, since gas is normally not accompanied by any liquid discharge. The bulk of a pad then, is simply not useful for gas odor. More importantly, the thick pad, and particularly the viscous glycerin, comprise a barrier to gas entering the pad. Gas, which seeks the path of least resistance, will simply by-pass and go around, rather than into the thick dense pad. Yabrov '417 claims “a deodorant including glycerin” in column 6, line 18 of the patent (6-18), “which absorbs hydrogen sulfide gas” (abstract). The chemicals responsible for odor in human gas are primarily indole and skatole, with perhaps small amounts of the mercaptans, butyric acid, valeric acid, or some amounts of certain other organic chemicals, but not hydrogen sulfide gas. Glycerin will simply not be effective for deodorizing human gas.

Weimer '398 describes an airtight polyurethane-coated nylon undergarment with elastic sewn around the waist and leg openings, and with a gas filter at a gas exit hole. Weimer identifies those suffering with several specific diseases as the beneficiaries of this invention in his “Background” section of the patent. Such an undergarment will be uncomfortable, hot, and sweaty, so patients must be suffering indeed, to tolerate wearing such underwear. However, several factors eliminate this product from consideration by healthy adults, for removing odor from normal gas in everyday life. First, healthy adults probably will not subject themselves to daily wearing of such punishing clothing. The characteristic plastic rustling noise (similar to baby training pants) made by anyone wearing a garment made of this particular fabric will certainly constitute an unacceptable social stigma to any normal active adult. The active ingredient for eliminating odor is a “single layer of activated charcoal on an open cell foam base” (2-45) having a thickness of ¼″, within a sewn pad. Experiments conducted by the present inventor has shown that a three-inch thickness of this form of the active ingredient is only briefly effective at eliminating odor from gas. In Weimer's device, the gas has only the ¼″ thickness of active ingredient to do it's work before the gas exits the filter. This form and thickness of carbon will result in a very short effective filter life. Further, because of the size and location of the filter in the garment, either in sitting or standing positions, will always block some areas of the filter, other areas of the filter will be preferentially overused and exhausted. The shallow ¼″ thickness will become exhausted fairly quickly, so the overused areas of the filter will be allowing unfiltered odor to pass therethrough fairly early, while previously blocked areas of the filter will be working well, and at the same time some blocked areas will not be filtering at all. The result will be poor filter performance, characterized by a long time period during which some unfiltered odor gets through the filter, with the odor gradually worsening over time. This may be a big improvement for persons with the listed illnesses described in Weimer '398, but healthy adults at work or in other social situations are seeking a sustained duration of complete elimination of odor.

Grosse '081 describes a shaped sanitary pad with permeable chambers containing granulated charcoal, for attenuating sound and absorbing odor from gas. There are problems with this invention which render it inoperable for daily use by normal active adults. First, the Grosse invention may work in a nursing home application, for example, a situation in which patients are normally seated, and the continuously seated position is holding the pad in a position in which it may function as intended. It is not apparent how the pad can be made to function as intended in any but a seated position, wherein partial weight of the body holds the triangular “insert 14” into “wedging positioning” (3-29). The wide configuration of pad member 12 will force insert 14 out of the wedging position as soon as seated body weight is removed, as when standing up, walking, etc. When insert 14 is not in a wedging position, this invention will fail to operate for the same reason as the other pad constructions discussed above: gas will take the path of least resistance and by-pass the pad completely. As one skilled in the art of construction, it is difficult to envision an “equivalent constructions” (2-19) or “suitable modifications” (4-55) falling within the scope of the Grosse invention, which enable this problem to be overcome. Grosse does not provide means for holding the device in a wedging position. If other conventional pad attachment means are used, such as fastening it to clothing with adhesive, pockets, straps, etc., these clothing attachments will only help dislodge the device from wedging position, as clothing shifts position during standing or moving about. It appears that a complete redesign, outside the scope of the invention, would be required to overcome this fundamental problem. In addition, the thick absorbent pad portion may absorb noise, but it requires greatly increased gas pressure to force the gas through the thick pad and through the granulated charcoal chambers. These impediments simply increase the likelihood that gas pressure will find an easier route past the pad rather than through it, by-passing the pad altogether. This will certainly be the case in any position other than seated position. Again, the social stigma and discomfort of having to wear and sit on a bulky pad stuffed into the back of one's underwear will be unacceptable to nearly all active adults.

The device disclosed in Conant '371 addresses the problem of gas odor for normal adults. Conant '317 eliminates the unnecessarily bulky pad employed by others, and uses the right active ingredient (activated charcoal, also called activated carbon). He employs a thin cloth form of charcoal, which is sufficiently inconspicuous such that social stigma is unlikely to occur. Unfortunately, his use of activated charcoal fails to solve critical problems. Some odor molecules are too large for adsorption by a cloth form of activated charcoal. Conant writes that “ . . . activated charcoal cloth is 100% activated charcoal . . . and adsorbs more effectively than granular forms of activated charcoal due to it's microporous character and higher internal surface area . . . ” (3-55). This statement can only be true if “more effective” refers to the speed of adsorption of uniformly small molecules only, such as would be a characteristic of a nerve gas or other chemical warfare agents, the purpose for which the cloth was developed. All cloth forms of activated charcoal begin with rayon fiber precursor material, and result (after activation) in a fabric having only uniformly small pore diameters, which are not capable of adsorbing any of the larger molecules. Thus, larger odor molecules may pass through activated cloth unaffected. If larger molecules must be removed, other non-cloth forms of activated charcoal must be used, such as coconut shell activated carbon which has a range of pore diameters, or coal or wood, which have still larger ranges of pore diameters. The present inventor's research has shown that the odor molecules in human gas definitely include some larger molecules which activated charcoal cloth pores cannot adsorb, even while smaller odor molecules are being adsorbed by the cloth. These larger odor molecules pass completely through activated charcoal cloth unimpeded, and deliver a portion of the original odor as an unfiltered bad odor even from the beginning of cloth filter use. This fundamental problem of charcoal cloth, makes it a particularly ineffective choice as an odor filtering media for human gas odor. One thin ply of such a material does not provide sufficient activated charcoal to be operable for its intended use. One single ply of charcoal cloth is so thin (both the knitted and woven types of charcoal fabric are ˜0.01″ thick), that its effectiveness in filtering substantial odor molecules from human gas is questionable. The Conant invention functions only when gas travels through the 1/100th of an inch thickness of the charcoal cloth, so the exposure time of the gas to the activated charcoal is extremely brief. Odor stripping opportunity will often be ended prematurely, as odorous gas abruptly exits the cloth thickness, still carrying odor. The obvious approach of adding more plies of charcoal cloth is not a practical solution because of the high cost of the cloth form of activated charcoal.

Inefficient filter design will necessitate premature pad changing The design of Conant '317 will cause gas to travel preferentially through only some areas of the cloth, due to seated position, or because some areas of the activated cloth are blocked. The preferentially used areas of thin activated cloth will become quickly saturated with adsorbed molecules and no longer adsorb odiferous molecules. This will result, for all subsequent odor molecules proceeding through the preferential areas, to pass completely through the cloth rather than becoming adsorbed. Seated-position temporary blocking will occur every time the user sits down, but some blocked areas will subsequently become available when the user stands or walks, and permanently blocked areas will be under utilized or never utilized in filtering. The frequency of filter changing will be determined by the generation of preferentially used (i.e., saturated) areas.

Permanent blockage of gaseous flow occurs wherever the double-sided tape is placed (6-41), as illustrated in Conant's drawing (FIG. 7) is present. All areas of the cloth directly underlying the tape are completely blocked from gas movement through the cloth thickness, and the tape completely eliminates the use of these cloth sections which are eliminated from participating in the intended odor-filtering function of the pad. The ineffective portion of the filter amounts to about half the total surface area of the charcoal cloth, according to the illustration. The overlaid “cloth layers or laminations” (6-17) also function as barriers themselves, partially preventing flow of the gas through to the charcoal cloth, except through the multiple perforations.

It may be possible to change the design within the scope of the invention, to help alleviate (but not eliminate) these blocking defects, but clearly any other tape configuration will inevitably be more complicated, and will always involve some degree of filter blocking. Other lesser-blocking attachment means, such as belts, straps, buttons, snaps, sewn pockets, velcro patches, etc, all require certainly uncomfortable and specially prepared underwear, and constitute a complexity clearly rejected by the scope of the invention (3-63). The perforated cloth laminations on either side of the charcoal cloth cannot be completely eliminated, since they perform essential practical functions, such as: (a) holding the charcoal layer in an out-spread position, as it is a very drooping type of fabric which will hang down and wad up otherwise; and (b) help keep the charcoal cloth intact through use and multiple washings, as it is fragile compared to other fabrics, and quickly disintegrates in normal pad-use conditions. Further, many normal conditions allow gas to by-pass the pad. Gas under pressure will always follow the path of least resistance, and relatively low pressure human gas is no exception. The charcoal cloth, by itself, will present a more resistant barrier to gas pressure than the spaces around it, especially if the cloth is not held tightly against the body. When additional obstructions are added to the charcoal cloth, such as the top and bottom fabric laminations, and double sided tape, the charcoal cloth pad becomes a substantial barrier to gas penetration, especially if the perimeter of the pad is loose, and not pressed tightly against the body. Unless the user's underwear is exceptionally tight, the pad can be expected to be loosely held against the body, particularly in standing or walking positions. Even if the pad is pressed tightly against the body, natural front and back cleavages and folds of the skin, and even hair, will present alternate paths of low resistance enabling gas to bypass the pad. Thus, loose clothing, pad thickness, and body contours and conditions will allow gas to by-pass the pad, rendering it inoperative in many instances.

In addition to the above-recited shortcomings, brittle charcoal cloth is impractical for reusability. The only reason for using the cloth form of activated charcoal is to provide the activated charcoal in a flexible, compliant form. This flexibility however, is a relative term. Carbon is brittle, and the cloth form of activated charcoal is notoriously delicate, fragile, and tends to disintegrate when subject to flexing and movement. Conditions for use of this pad, and especially the washing conditions, are extremely harsh for a brittle cloth such as this. The pads can be expected to have a relatively short useful life before tears and holes in the charcoal cloth provide passages for unfiltered gas to pass through the fabric, and require early disposal. It is important to note that the chemical warfare cartridge applications for which the charcoal cloth was developed, always dispose the cloth in a fixed, non-moving position, which accommodates the inherently brittle property of charcoal cloth.

Reusing dirty pads is unsanitary and unlikely to be accepted. For purposes of cleanliness, such pads should not be reusable. Cleanliness aside, practicality alone would dictate disposability. For example, if multiple pads were used in a single work shift, how would the dirty pads be stored and brought home for cleaning? More readily than the other forms of activated carbon, the cloth form of activated charcoal always quickly seeks a state of equilibrium, so a dirty pad exposed to cleaner air, will reverse the process of adsorption, and quickly desorp it's accumulated odor molecules into it's new clean air environment. Consequently, the dirty pads will be giving off high concentrations of odor whenever handled, and wherever they are stored at the work place and on the way home. It seems obvious, that people will not want to handle these dirty pads, or wash them. In fact, the idea of cleaning and reusing these pads will probably be repugnant to most users.

A principle reason for reusing such carbon fabric-based pads is to absorb the high cost of activated charcoal cloth, by spreading the expense of each pad over several uses. Pica USA, a supplier of “Actitex” woven activated charcoal fabric, quotes charcoal cloth at $100.00 per sq. yd, and a new lower grade (not tested) was said to be about $30.00 per sq. yd. Based upon FIG. 5 and FIG. 6 in the patent drawings, a yield of about 30 die-cut pieces of fabric can be cut from a square yard of cloth. This amounts to a material cost of from $3.34 each to $1.00 each for the die-cut charcoal cloth alone. Recognizing that adhesively bonded laminations would create additional gas flow barriers and further block gas flow through the pad, it is assumed that the two fabric laminations would be sewn to the carbon cloth and require a sewn edge to prevent fraying. After die tool amortization, cost of all other materials, labor, packaging, distribution costs, shipping, and profit margins are added, even at the low-priced charcoal fabric cost, each pad can easily cost over $4.00 each. To be disposable, this type product must cost less than $2.00 each, which is a price point unachievable for a Conant-type pad, precluding the pad's reusability.

Thongs, or thong-type undergarments are well known in the art. For example, three thong-type, non-deodorizing sanitary napkins specifically adapted for thong-type underwear are disclosed by Darby (U.S. Pat. No. 5,683,373), Sturino (U.S. Pat. No. 5,713,886), and Williams (U.S. Pat. No. 5,729,835). None of these involve any kind of gas odor removal mechanism, nor do they relate in any way to filtering odor molecules from a gas stream. In summary, there are problems associated with prior art filtering devices that fall into two main categories: (a) inefficient carbon utilization; and (b) filter by-pass. The present invention cures these deficiencies.

### SUMMARY

It is an object of the present invention to provide a gas filtering device adapted to be worn upon the body and operable for receiving a gas stream bearing odiferous molecules emanating from the body, thereafter removing substantially all odiferous molecules from the gas stream and releasing a substantially odor free gas stream.

It is a further object of the present invention to provide a gas filtering device comprising an odor adsorbing element wherein the filtering device retains its operability for removing odiferous molecules from a gas stream until the odor adsorbing capability of the entire odor adsorbing component is essentially exhausted.

It is yet a further object of the present invention to provide a gas filtering device adapted to be worn upon the body and operable for receiving a gas stream bearing odiferous molecules emanating from the body, wherein substantially the entire gas stream emanating from the body passes through the odor adsorbing element comprising the gas filtering device.

It is still a further object of the invention to provide a gas filtering device meeting the above objectives that is inexpensive to manufacture, disposable, has a low profile and includes attachment means thereon operable for releasably attaching the gas filtering device to an undergarment.

Another object of the present invention is to provide a gas filtering device and a method for using the device to reduce or eliminate odor filter by-pass due to the explosive emission of a large bolus of gas from the body.

The above objectives of the present invention are met by incorporating one or more linear matrix filters into a thong. Linear matrix filters provide alternate gas exit paths, and this process effectively sub-divides the total gas volume passing through the filter by the total number of equivalent linear matrix filters provided. In accordance with the principles of the present invention, the objective of overcoming excessive gas volume by-pass is accomplished by providing the filter with a high air permeability gas entry site in direct contact with the body's gas exit location, and by providing a filter with enhanced gas flow characteristics.

Another object of the present invention is the provision of a method for overcoming odor filter by-pass due to gas flow blocking factors, such as body-position blocking (sitting down, for example) of gas routes through the filter. In accordance to the principles of the present invention, the objective of overcoming blocking factors is accomplished by the method of providing commonly vented alternative gas filters. One method for example, provides two gas filters with a common gas input site, which are normally both used equally and simultaneously. If changing body-position of the user should happen to temporarily block one filter, then all gas will automatically shift to exiting out of the alternative filter, until another shifting of body position opens up the blocked filter, and both are used simultaneously.

Another object of the present invention is the provision of a method for overcoming problems associated with the attenuation or muffling of the sound of escaping gas. In accordance to the principles of the present invention, the objective of overcoming sound muffling problems is accomplished by placing high air-permeability materials in direct contact with the gas exit location, thereby eliminating the vibrations which cause gas sound while simultaneously providing unobstructed gas filtration.

Another object of the present invention is the provision of a device which overcomes all the problems associated with conventional attachment devices. In accordance with the principles of the present invention, the objective of eliminating conventional attachment device problems is accomplished by providing a surface area contact attachment method, which holds the gas filter securely in place by maximizing skin-contacting surface area, filter location, correct selection of skin contacting materials, and body-compliant ergonomic configuration.

Another object of the present invention is the provision of a method for admitting gas into, and controlling gas flow through and out of, the porous gas filter internal construction. In accordance with the principles of the present invention, the objective of providing controlled gas entry and gas flow through and out of the filter is accomplished by the method of providing an otherwise gas impermeable external membrane on the filter, with openings in the membrane for gas entry and gas exit.

Another object of the present invention is the provision of a method for providing gas exit routing away from the filter, so that blockages of gas flow that occur after exiting the filter do not prevent the filter from functioning. In accordance with the principles of the present invention, the objective of providing gas routing away from the filter is accomplished by the method of porous multi-folded positioning straps that converge at the gas exit sites on the filter.

Another object of the present invention is the provision of a method for constructing a gas odor filter to be used by both men and women, and which accommodates a woman's use of conventional feminine pads. In accordance with the principles of the present invention, this is accomplished by provision of the “thong method”, which accommodates both sexes, but which provides ample room in the front of the thong for placement of conventional feminine pads, by aligning the front positioning strap on one side of the crotch.

The present invention discloses an odor filtering device operable for removing odiferous molecules from a gas stream exiting a body through an orifice in the body comprising: (a) a filter comprising activated carbon, the filter having open ends providing a plurality of gas exit ports and a body portion therebetween, wherein a centrally located portion of the body portion comprises a gas input port; (b) a barrier film that is substantially impermeable to the gas, the barrier film enveloping the body portion of the filter but not the open ends of the filter. The barrier film has a hole therein disposed to overlie the gas input port on the body portion of the filter. A gas permeable fabric overlies the both the barrier film and the hole in the barrier film. The filter, barrier film and fabric, in combination, form an elongate filtering device having integral construction.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1(a) is a perspective view of a linear matrix filter containing activated carbon, with enlarged portions of the filter showing odor-laden gas entering the matrix (FIG. 1(b)), and clean, odor-stripped gas emerging from the matrix (FIG. 1(c)).

FIG. 2 is a schematic view illustrating the operation of the “Safe-T” configuration of a multiple matrix filter, showing the position of the filter against the body. An enlarged view of the portion of the filter in contact with the gas exit opening on the body is shown in FIG. 2a.

FIG. 3 is a cut-away side view of the Safe-T filter, showing underlying construction at the common gas entry manifold location, and gas direction through the filter.

FIG. 4a-c illustrates construction of a Safe-T filter.

FIG. 5a-e illustrates some variations of a Safe-T filter matrix material configuration.

FIG. 6a-d illustrates some alternative multiple matrix filter embodiments.

FIG. 7a-b is another alternative multiple matrix filter embodiment.

FIG. 8 is a partially cut-away view of an odor filtering device in accordance with a particularly preferred embodiment of the present invention.

### DESCRIPTION OF THE PREFERRED EMBODIMENTS

Linear Matrix Process of Using Activated Carbon to Remove Odor from a Gas Stream

Activated carbon filtration methods often have a stubborn inefficiency problem: some areas of a filter become preferentially exhausted due to various reasons, and the exhausted sections begin to allow unfiltered materials through, which necessitates filter replacement before all of the carbon in the filter is exhausted. The formation of exhausted portions of the filter require replacing the entire filter, even while substantial portions of the filter remain able to function effectively. The activated charcoal cloth in the Conant invention for example, as reviewed above in the discussion of the prior art, suffers from this problem when preferentially over-used sections of the cloth become quickly filled and no longer adsorb odor molecules. This results in all subsequent odor molecules which are proceeding through those preferential areas, to pass completely through rather than becoming adsorbed. Meanwhile preferentially under-used sections are still virtually or completely clean. When odor escapes through the over-used sections of the filter and is smelled by the user, the filter is perceived by the user as “used up”, and ready to be changed. In reality, perhaps as little as 10% of the activated charcoal cloth may be used up (i.e., saturated with odiferous molecules adsorbed from the gas stream), but the 90% un-used areas of cloth will be replaced along with the 10% used-up areas which are causing the problem by allowing odor through the filter. This inefficiency is overcome by the “Linear Matrix” process, which insures that virtually all of the activated carbon has adsorbed all the odor it can (i.e., is saturated), before any smell is detected, indicating to the user that filter replacement is required.

Description of the Linear Matrix Process.

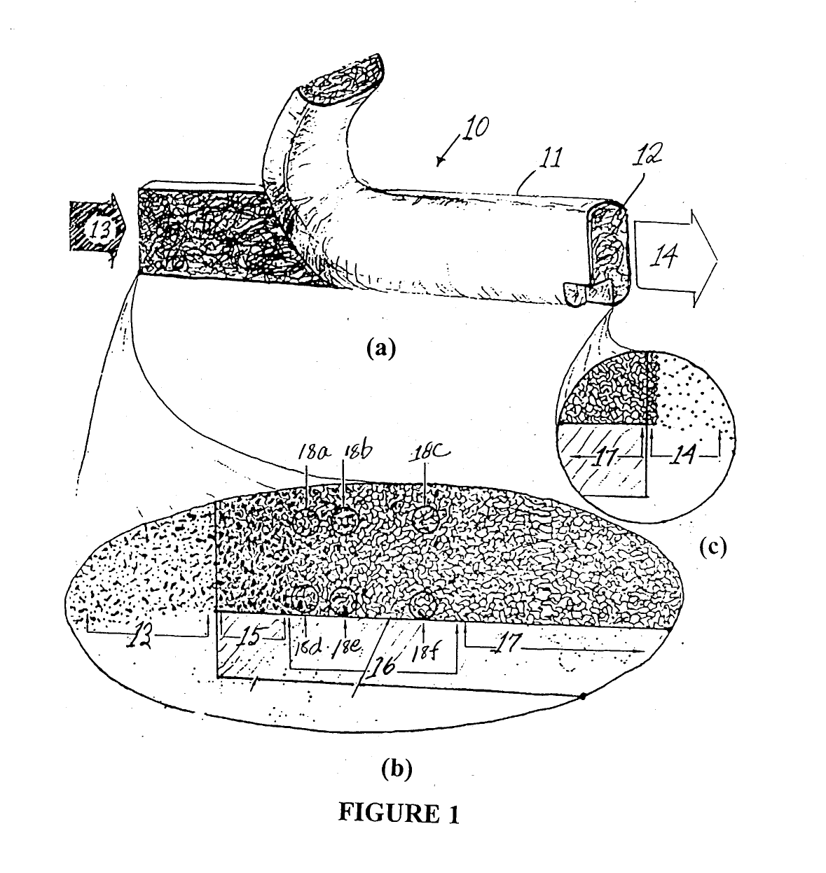

FIG. 1 is a diagram of a cut-away linear matrix filter containing activated carbon 10 with a gas barrier 11, a matrix 12, odor-laden “dirty” input gas 13, and odor-stripped “clean” output gas 14. Entering the matrix 12 from the left, as indicated by the broad arrow, is an odor-carrying gas 13 which passes through the linear carbon matrix 12. Odor molecules comprising the gas input stream 13 are very large organic assemblies, relative to the very small gas stream molecules. The odor-laden gas 13 entering the filter 10 is essentially at the same pressure and velocity across the diameter of the matrix, and consequently, will deposit odor molecules more or less uniformly across the diameter of the matrix as it progresses from left to right through the length of the matrix. This uniformity of odor molecule deposition manifests itself as essentially the same quantity of odor molecules trapped within carbon cavities between any two sites across any given diameter of the matrix. Thus, the concentration of odiferous, trapped molecules in portions of the filter indicated at numerals 18a, 18b and 18c will be the same as the concentration of odiferous molecules in portions 18d, 18e and 18f respectively (FIG. 1(b)). The concentration of odiferous molecules trapped within carbon cavities (in the direction of gas flow) decreases between any two sites along the length of the matrix 12 within the “high activity odor fill zone” 15 (FIG. 1(b)). The fill zone 16 is located between the essentially odor-filled carbon cavity input section of the matrix length 15, and the essentially nonutilized carbon cavities of the linear output section 17 of the matrix 12. Thus we can see that any given large odor molecule coming in close proximity to cavities of the right size (roughly three or more times the diameter of the odor molecule), and sometimes with the help of existing slight surface charges, the odor molecule will soon fall into (and under some conditions partly be attracted by surface charges into) a cavity, and get trapped therein. As a gas with odor molecules passes through the torturous paths in a matrix 12, passing within close proximity of activated carbon cavities, the gas becomes stripped of odor molecules, and emerges odor-free from the carbon filter 10 as indicated at broad arrow 14.

The explanation that follows, with a description of three distinct zones, actually occurs within a specific cavity size range for each group of molecular sizes. But the fill zones are of different lengths and molecules comprising the gas stream 13 progressively fill cavities through the filter at different rates for each of the different molecular size groups, according to size availability of cavities in the carbon, and proportional number of odor molecules in the odor mix. In reality, all sizes of cavities are filling at the same time, with the three zones for each molecule size groups all superimposed upon each other. But, if we grouped all the odor molecules by size, and sent one molecule size at a time through the filter, we can clearly understand that each group of molecules segregated by size will all have the same three zones, as molecules fill cavities in the filter and the zones move through the filter in the same way, as they fill characteristic cavity sizes. The order inherent in this process of cavities filling with odor molecules, will be consistent from one size to the next. For clarity, it is helpful to look at one group of similar sized odor molecules passing through the filter 10.

With reference to FIG. 1(b), which illustrates a Linear Matrix filter, within the carbon matrix length, we can distinguish three distinct areas: (a) the “essentially full zone” 15, where a previous gas volume was stripped of it's odor molecules; (b) the “high activity filling zone” 16, where gas currently progressing through the filter is or will soon be stripped of it's odor molecules; and (c) the “essentially empty zone” 17, where subsequent volumes of gas 13 passing through the filter 10 will be stripped of it's odor molecules in the future, and where a present gas volume that was already stripped of it's odor molecules in zone 16, is currently a clean gas passing through this zone without any odor molecules being deposited in this portion of the matrix. As the first-exposed carbon cavities in zone 15 fill up with odor molecules, subsequent dirty gas rushes past the full cavities unaffected, only to deposit their odor molecules into unfilled cavities farther along the carbon matrix in zone 16. The carbon cavities are being essentially filled up, beginning at the input end of the matrix, with the filling zone moving along the length of the matrix, all the way to the output end in empty zone 17 of the matrix. Beginning at the input end of the matrix, the full zone 15, which starts as 0% of the matrix length (before use), can grow to 100% of the matrix length as filtration capacity of the filter is exhausted, at which time the filter is completely exhausted and must be replaced. The second zone is the filling zone 16, in which most of the cavity-filling/odor stripping activity occurs. This zone separates essentially unreceptive full cavities in zone 15, from essentially receptive empty cavities in zone 17. The filling zone 16 starts as 0% of the matrix length (before use), and, with use, maintains a fairly stable length (which length varies, depending upon several factors such as type and amount of carbon, mix of odor molecule sizes, density of carbon cavities of each size category, temperature, etc). The filling zone 16 travels through the matrix length, from the input end to the output end, as odor molecules fill up the carbon cavities. As this zone reaches the output end of zone 17 of the filter, unstrapped odor molecules from this zone begins to emerge from the output end, and continue to emerge with increasing frequency as the zone 16 shrinks to 0% of the matrix length. The empty zone 17, which starts at 100% of the matrix length (before use) and shrinks to 0% of the matrix length just as the fill zone 16 reaches the output end of zone 17, and zone 17 disappears and the filter 10 begins to let some odor through. During filter use, the filling zone 16 advances through the matrix, as cavities fill with odor molecules, from the input to the output end of the carbon matrix, along the gas stream path. The filling zone moves farther and farther along the length of the matrix, as the gas stream carries it's burden of odor molecules into the matrix, past the already filled cavities, and on to the awaiting empty cavities farther along the gas stream path. Meanwhile, the linear matrix is continuously producing a clean gas that emerges from the output end of the matrix, stripped of odor molecules. Eventually, and relatively abruptly, the fill zone will reach the output end of the matrix, which means that some odiferous gas is now passing completely through the matrix without encountering sufficient empty cavities for odor molecules to become trapped within. If the filter continues to be used, these odor molecules begin to emerge in growing number along with the gas molecules, from the essentially completely filled matrix, which indicates that the activated carbon in the column has virtually exhausted it's odor trapping capability, and must be replaced. If the filter continues to be used, the number of odor molecules emerging from the output end will progressively increase until the fill zone 16 length shrinks to 0% of the matrix length. At this point, as the odor trapping cavities for this size molecule are effectively filled, nearly all odor molecules of this size will travel completely through the filter and out through the output end.

Since there is never a perfect proportional balance of odor molecule quantity to cavity quantity for each size of odor molecule in any particular odor/gas mix, there will be a period at the end of the filter life when all cavities for certain sized odor molecules are completely filled, but some empty cavities remain that continue to trap all odor molecules of certain other sizes. During this period, the odor molecules of the size whose trapping cavities in the carbon are completely filled, will go right through the filter. At the same time, certain other sized odor molecules whose trapping cavity sizes are not completely filled, will continue to trap and filter out all those correspondingly sized odor molecules. The result will be a period in which the filter does not completely eliminate all odor, but allows a changed, partial odor through, comprised at first entirely of one size of molecule, based entirely on molecule size and availability of carbon cavities per each size. The odor emanating from the empty zone 17 of the filter will be very different from the odor mix entering the full zone 15 of the filter at the input end. It will be faint and unrecognizable at first. As other sizes of cavities fill up and allow other sized molecules through, the mix of molecules emanating from the filter will become more like the unfiltered mix of molecules at the input end. As an increasing number of molecules of each size emerges from the filter, the odor will get stronger. At first, there will be a brief period in which an emerging odd odor will be faint, noticeable, unrecognizable, and inoffensive. This brief period is the opportune time to change the filter, since this “indicator smell” (which will not be perceived as a bad odor) provides an indication that the filter is on the verge of being completely consumed, and will soon be allowing bad odor through the filter. When all the filter cavities of all the sizes are essentially full, virtually all odor molecules entering the filter will pass through and out of the filter, with no perceivable difference in the bad odor.

Advantages of Linear Matrix Process—Because the linear carbon matrix controls the gas flow so that it uniformly enters one end only of the linear carbon matrix, and forces the gas stream to emerge from the opposite end only of the linear matrix, the matrix is the most efficient carbon filter possible. It provides a filter which will be useful as a filter until it becomes thoroughly exhausted of its odor trapping capability. The matrix is also a filter which can only produce a clean gas stream from the output end of the filter, until it's filtering capabilities are fully exhausted, and the emerging gas rapidly changes to a dirty gas stream just as the matrix is exhausted due to saturation of the cavities comprising the matrix. The linear carbon matrix filter provides an odor filter with a predictable lifetime, which produces an inoffensive odor “change indicator” just before the complete exhaustion of that lifetime.

Multiple-Matrix Process

Several examples of prior art gas filters have a common problem of gas bypass. This occurs when the filter experiences an abrupt excessive volume of gas which exceeds the filter's capability to accept and filter the larger volume of gas at one time. Excess gas will escape by going around the filter along the path of least resistance. The best way to prevent by-pass, is to provide a filter wherein the path of least resistance is through the filter. Doubling this path by creating an alternate filter, provides a “safety valve” that reduces the resistance to gas flow and substantially doubles the gas filtering capacity in the event that the gas volume to be filtered exceeds the capability of one filter, and provides an independent exit path in the event that one route becomes blocked.

The present invention provides a multiple-matrix method for reducing or eliminating filter by-pass. This method provides multiple linear matrix filters (or the equivalent of multiple-matrix filters) having a common input manifold which function as separate gas exit conduits to accommodate greater gas volume and also function as an alternative gas flow route if blockage occurs. By supplying a filter comprising multiple linear matrix subfilters with a stream of gas from a common manifold, it enables the subdivision of the gas volume into a plurality of parallel subfilters, the number of such subfilters based upon each filter's gas flow capacity, and allows multiplying of the gas volume accommodated by one matrix filter. A multiple matrix filter accepts larger gas volumes through the manifold, rather than causing excess volume to build pressure until a portion of the gas escapes around the filter, thus by-passing the odor filtration process.

The human body provides two convenient alternative locations for placing a pair of linear matrix filters, wherein the gas exiting the body in both these directions simultaneously, forms a path in the shape of an inverted “T”. An embodiment of a filter 10 that comprises two linear matrix subfilters is referred to herein as the “Safe-T” embodiment of a filter in accordance with the present invention.

Description of the Safe-T Preferred Embodiment of the Multiple Matrix Process.

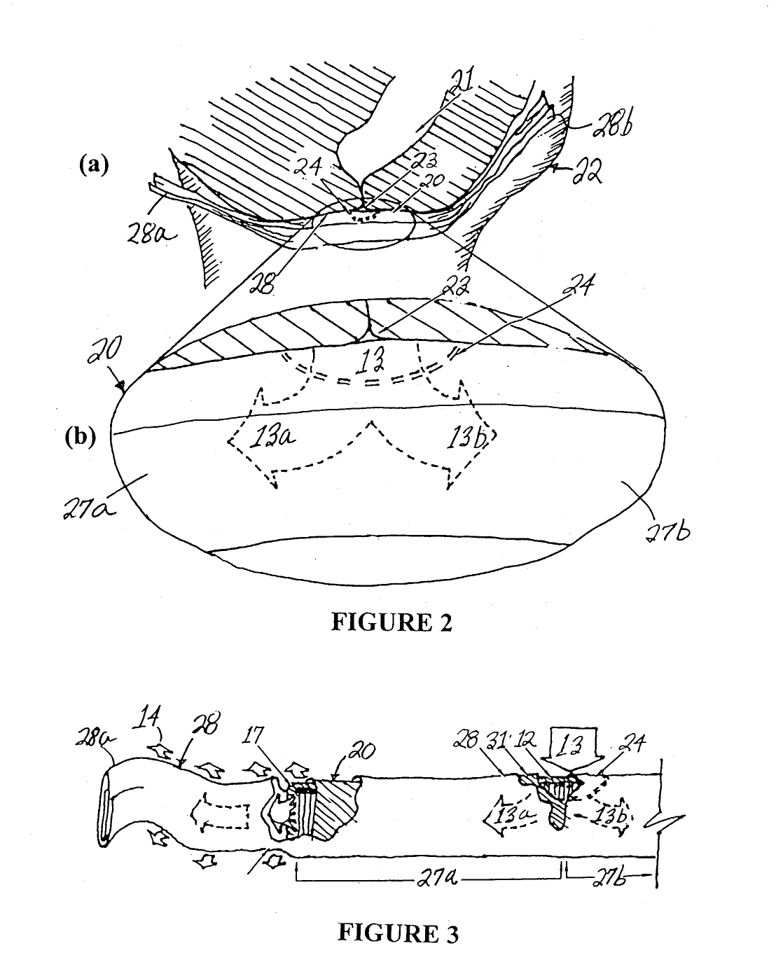

FIG. 2(a) and enlarged FIG. 2(b) are schematic diagrams illustrating the operation of a Safe-T filter 20. FIG. 2(a) and, in greater detail, FIG. 2(b) show how the multiple matrix linear gas filter 20 accepts a perpendicular gas stream 13 through a central entry location 24 of the filter, and channels the gas stream into one of two oppositely directed linear matrix devices 27a and 27b, each of which accepts half the volume of gas 13 entering the filter. This doubling of the exit conduits effectively divides the total gas volume by half for each linear matrix, with only half the total gas volume exiting through each matrix subfilter. Gas 13, generated in the human digestive tract 21, exits the body 22 through the anus 23, venting directly into the filter at a “manifold” area 24 (within the double dashed line), disposed at the center and top of the filter 20. Gas 13 entering the manifold will divide to flow into one of two easy exit routes 26a and 26b through the filter 20. These routes pass through the two matrix filters 27a and 27b, which are joined end-to-end, with a common gas input manifold 24. In use, the filter 20 is encased within, and supported by, a thong 28 comprising a porous fabric tube having “tails” 28a and 28b adapted to releasably engage an undergarment (not shown) to hold the aperture 24 of the filter adjacent the anus 23 as will be discussed below.

FIG. 3 is a cut-away view of the filter, shows internal details of the filter. In the cut-away view, unfiltered gas 13 entering the manifold area 24 within double dotted line, which manifold 24 sits like a saddle on the filter, will first pass through the porous fabric cover 28, then through a large hole in a barrier film layer 31 underlying the fabric cover 28, and enters the activated carbon matrix 12 comprising the filter 20. The gas makes a 90 degree turn into one of two opposite directions 13a and 13b, and follows any number of torturous paths through the filter media, at which time the odor molecules are stripped from the gas stream, as described above. The barrier film 31 prevents any gas from escaping the filter media until the gas 13 reaches the end of the empty zone 17 of the matrix 12. Filtered gas 14 exiting the output end of the filter enters the porous fabric tubing 28, scrubbed of odiferous molecules. From this location, clean gas 14 will proceed along the folds of the cloth tubing until it escapes through the pores therein along the length of the tube, or out the open end of the porous tubing 28.

Multiple-Matrix Filter Material Testing

Many different materials can be used to construct linear matrix filters in several different configurations, all of which function adequately. Extensive testing has identified the preferred overall designs. Fundamentally, there are only three basic materials needed: (1) a filtering media comprising activated carbon; (2) a barrier film, which wraps over the filtering media to block gas flow, except at the gas input and gas output openings in the film; and (3) a gas permeable or porous fabric tubing which contains the filter medium and/or provides a covering for the filter and performs several functions.

Media Pre-Screening—Various media materials have been evaluated. A prescreening of all forms of activated carbon eliminated some materials that were not available in a functional form. To be functional, the commercial form of the filtering medium had to be clean, and minimally breathable under “application compaction”, which means gas will still adequately pass through the media after long term compaction, such as 8 to 10 hours of use by a tester at a desk job. This requirement eliminated some known materials from testing, such as all bulk powder forms of bituminous, wood, and coconut shell (cheaper, but not breathable in an off-the-shelf form, plus presents a clean-up problem if the filtering device breaks open).

Media Test Apparatus—The test apparatus used was very different from a conventional “weight gain” test, in which unlimited exposure to a single known gas is performed under controlled conditions, with percent of carbon weight gain indicating media absorption of that particular gas. This standard test would not reveal the most relevant data, such as what volume of odiferous gas can be cleaned before filter failure starts, or whether the onset of odor getting through was of the “whole odor” or only a portion of the complex odor mixture, and if a portion, then which category of gas constituents were getting through the filter first, and at what point was the odor objectionable, etc. Clearly, controlled conditions and actual application odor must be used for testing, not simplified “representative” single gases or gas mixtures.

Media Testing—Five different media were tested in the new apparatus, developed to simulate the actual odor exposure conditions which the media would be subjected to in application. These were: (1) Reticulated foam impregnated with activated coconut shell carbon @ ˜$3.00/sf; (2) Synthetic fiber mesh impregnated with activated coconut shell carbon @ ˜$1.00/sf ($250 minimum order); (3) Extruded activated coconut shell carbon particulate between two thin polyester scrims @ >$3.00/sf, which also represents loose particulate at lower cost; (4) Knitted rayon cloth activated carbon @ from $11.00/sf to $3.30/sf; and (5) Rayon felt activated carbon @ ˜$2.50/sf.

Test results were as follows:

Media Material/Test Amount/Slight Odor/Bad Odor/Test Stopped/Clean Volume/Grade.

1. Foam/1.5 grams/300 ml./406 ml./406 ml. (bad odor)/<1.0 liter/poor

2. Mesh/1.2 grams/never/never/6,050 ml. (no odor)/>6.0 liters/excellent

2a. Mesh (retest)/1.3 grams/never/never/17,406 ml. (no odor)/>17.4 liters/excellent

3. Extruded/1.75 grams/never/never/17,406 ml. (fresh smell)/>17.4 liters/best

4. Cloth/1.6 grams/from the start/>4,800 ml./˜6,000 ml. (bad odor)/never clean/failed

5. Felt/1.2 grams/from the start/>3,600 ml./˜4,800 ml. (bad odor)/never clean/failed

Media Test Results—

[1] The cheapest materials (item #2; mesh, and #3 extruded—but in loose particulate form) were the two top performing materials.

[2] By filtering bad odor from more than 17 liters of air, the two best performing materials (mesh and extruded particulate) far exceeded anticipated service life requirements. Both materials are capable of more filtration since testing was arbitrarily stopped at this volume of about 17 liters.

[3] Cloth and felt both failed, probably because their well known “uniformly small size cavity range” could not filter out larger size molecules, which are apparently contained in bad odors of this type.

It is possible that sewage odor contains quantities of molecules of a size too large for cloth and felt carbon materials to adsorb. This is supported by two anecdotal observations. First, with both the Fabric and Felt tests, the odor components first to be passed through these materials were perceived to be “bottom notes”, to use the term of the perfumer, which characteristically are less volatile, larger molecular components of the gas. Second, prototype tests of Safe-T filters used very old Mesh media, which had lost most of it's adsorption capacity. These tests were very brief, and ended when the devices failed to filter certain odor. The first odor components admitted through these mesh filters were perceived to be top notes, which are more volatile, smaller molecule components. There are different filter failure modes characterized by the first molecular size of odiferous molecules emanating from the filter, and classifiable by smell into at least top or bottom note categories. While failure mode will be dependent upon both odor molecule mix and type of filtering medium, if a particular odor molecule mix produces a relatively very early failure mode in any particular media, this indicates which pore size there is a relative shortage of in that specific media.

Barrier Film Testing—Three materials were tested, and each met the two pre-screening functional requirements: the film must be a thin plastic film gas barrier, and not subject to excessive flexure cracking. Three materials were tested.

Film Material/Handling/Die cut/Clinging/Heat Seal/Cost/in-use/Overall

1. Sandwich wrap/difficult/poor/excellent/yes/very low/quiet/marginal

2. Stretch wrap/very difficult/poor/excellent/yes/low/quiet/marginal

3. Polyester/easy/good/not at all/yes/very high/quiet/marginal

Barrier Film Test Results—

[1] All three films will form functional film barriers, but all have problems that are not desirable long term, so other films should be evaluated. A flat film could be hot die stamped onto a release film continuous roll. Another alternative is to employ a heat shrink sleeve, which can be flattened and notched for the hole, and precut to length; or punched with a hole pattern for the saddle and perforated at lengths in a continuous sleeve to speed heat shrink installation.

[2] The polyester film was expected to have a problem with noise. Film hardness gave the bare film a loud “crackling” noise which would be objectionable. Surprisingly, in use, the film was silent. The fact that it was not a clinging film means that it folds and creases rather than consolidates, around the media. These folds could potentially become by-pass routes around the filter, which are unacceptable. Fortunately, the in-use testing did not produce this problem.

Fabric Pre-Screening

The porous fabric tubing used to make the prototype filtering devices were made with a “found” material which was available at the moment. It was a non-woven polyester packing fabric from Mexico, which was both difficult and ideal in different ways, but may be the best overall choice. Test materials had to have a high air permeability value (providing unobstructed gas flow through one ply of the fabric into the filtering medium), and yet be strong enough to withstand prolonged use. In addition, the fabric must be a heat sealable, low cost, non-woven fabric with a good “hand” to impart a comfortable feel to the skin. Numerous polyester and nylon candidate materials were examined as well as a polypropylene material.

Fabric Testing—The pre-screening narrowed the field of initial fabric candidates down to four:

Fabric Material/Type/Softness/Air Permeable/Heat Seal/Hand/Non-Slippage

1.Prolec; Monterey Mexico/˜1.5 osy polyester/marginal/˜250 cfm/ft²/marginal/good/marginal/excellent

2.PBN spun bound (Cerex)/1.0 osy nylon/excellent/602 cfm/ft²/excellent/very good/excellent/poor

3.PBN trilobal spunbound/1.5 osy nylon/good/606 cfm/ft²/marginal/very good/good/marginal

4.Cerex chem. low-bond/1.5 osy nylon/acceptable/250 cfm/ft²/marginal/very good/good/poor

Fabric Test Results

[1] While the Mexican fabric is marginal in three important areas, it has a good “tooth” which makes it excellent in the “non-slippage” category, and, at about one quarter the cost of Nylon, it remains the best material overall that was tested.

[2] The nylon did so well in all categories except the requirement for non-slippage, that it may be easier to overcome the slippage problem associated eith the nylon, than to accept the problems of the polyester. Two less-slick nylon materials were tested (items 3 and 4), and the trilobal was an improvement for nylon, but it may be possible to find an adhesive to apply to the sides of the nylon filter to accomplish this.

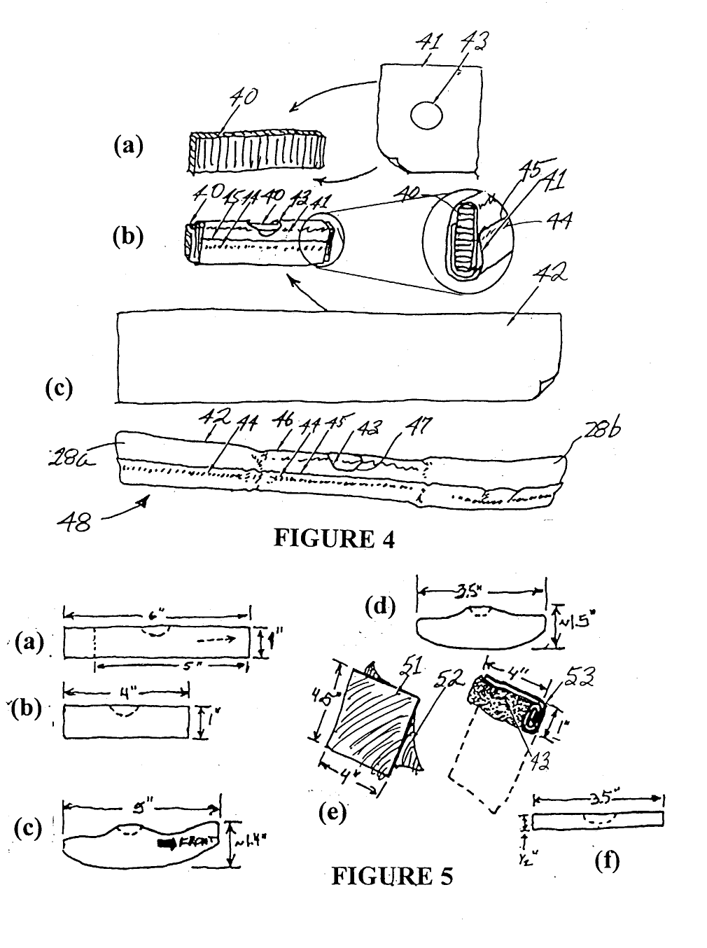

Preferred Filter Materials—New and better materials may become available at any time, and materials substitution for better performance is obvious. The results of testing to date show that generally, the Mesh and extruded particulate forms of carbon have performed the best of the tested materials for the filtering medium indicated at numeral 40 in FIG. 4(a). However, for certain applications one of the other media tested above may be used which satisfy one or more identified criteria, or a form of filtering medium not tested above, such as supported or loose-bagged activated carbon, or loose yarn for example, or any combinations of tested and untested carbon types may be used in any form with fillers, performance additives, or performance enhancers or extenders, such as metals, chemicals, or mixtures or compounds, applied to the carbon media as sprays, dips, coatings, mixtures, material mingling, etc. Combinations of carbons may be used to provide combined or synergistic benefits of the separate carbon ingredients. For example, one or more of the powdered or particulate carbons added within the open cells of reticulated foam or carbon coated reticulated foam, would gain the advantage of mixed carbon type absorption plus the resiliency and breath-ability of the foam. Another example would be lacing or interleaving layers of carbon particulate or powder types or mixtures of types of particulate or powder, with carbon cloth or felt or mesh layers, to achieve combined or synergistic benefits.

Similarly, the barrier film 41 used to cover the media 40 in FIGS. 4(a) and 4(b) may be any one of the tested single or multi-layer films, which will all work. Any number of other films categorized above, or film forms that meet one or more barrier film criteria identified above but were not specifically tested here, but which will perform the specified functions, will also work. Included would be any of the other heat shrink methods and materials, as well as hot die stamped films and sleeves, spray coatings, extruded coatings, carrier-sheet film wraps or transfers, powder coated heat or chemical consolidated coatings, etc.

As with the other two materials (i.e., the filter medium and the barrier film), the fabric top layer 42 shown in FIG. 4(c) may comprise any of the tested fabrics, or any of numerous non-woven alternatives which satisfy one or more fabric criteria identified above. Some obvious alternative materials include woven fabric scrims or thin cloth, fabric-like single or multiple layers that include cellulose or other paper-like water degradable sheet materials; and water degradable and biodegradable woven, non-woven, and plastic sheet materials.

Preferred Filter Embodiments

With reference to FIG. 4, illustrating the construction of the Safe-T filter embodiment and it's variants, a barrier film 41 has hole 43 cut therein. The film 41 encloses, by way of wrapping in this case, a strip or bag of filtering medium 40. The media/film assembly shown in FIG. 4(b) shows film 41 wrapped about the media strip 40, with film hole 43 centered along the length and width on a top edge of media 40, and the hole 43 disposed over the media strip 40. The media 40 is fully exposed through the hole 43. At both ends of the filtering medium strip 40, the film is about ¼″ too short to meet flush with the ends of the media. The film 41 is wrapped around the strip of filtering medium 40 in such a way that the film does not overlie the hole 43, but provides a double layer of film covering portions of the filter medium strip 40 but not the ends and the hole. When wrapped, the film may be secured by heat sealing 44 along at least the outermost film edge 45 at the side of the media strip 40. Other securing methods may be used, such as heat staking, ultrasonic staking, hot glue staking, chemical or solvent bonding, or taping along at least one film edge. Fabric material 42 provides the outer wrapped layer, applied directly over the film layer 41. The completed filter 46 (FIG. 4(c)) has fabric 42 wrapped snug around the media/film assembly, which fabric is secured to itself, secured to the film layer, and partly secured to the media by heat sealing 44, or other securing methods described above, along at least the outermost fabric edge 45. The ends 28a and 28b of the fabric 42 extending beyond the ends of the filtering medium strip 40 can form a strap at each end of the filter, and the free ends of the strap may be releasably affixed to an undergarment by temporary attachment means such as an adhesive, or other securing methods. The location of film hole 43 is indicated on the fabric directly above the hole, by any marking method, such as tracing, drawing, coloring, or printing, of the outline of the hole upon the fabric, as shown at 47. Within this outline 47, only one thin layer of fabric overlies the gas input location for the filter. The 3-dimensional volume underlying the outline 47 constitutes the gas input manifold, which accepts all gas which will travel through the filter 40.

Variations of Preferred Embodiments

The filter 40 and the device 48 comprising the filter 40 is flexible enough for a rectangular shaped filter to bend to the body contour and stay in place, but specialized configurations are anticipated. Some of these modifications may include designs similar to those shown in FIGS. 5(a)-(f). All designs shown in FIG. 5 depict the portion of the device 48 comprising the activated carbon media strip only, with the other portions and layers of the device 48 having been removed for clarity. The gas input manifold location 47, or “saddle” is shown on the media variations, indicated by the dotted line. The barrier film and the fabric layers will be selected using the results of material research above, and accompanying material identifications and observations as selection guidelines. These layers can be modified or adapted to accommodate the particular size and shape of the filter 40 shown in FIGS. 5(a)-(f). Some modified designs which have been built and tested:

Modification/Size/Weight/Lab Wt./Test multiple/Volume/Gen. Comfort/8 Hr. Sitting Test.

1.Mesh Maxi/1″×6″/2.8 g./1.3/2.15 times test/37+liter/Acceptable/Uncomfortable

2.Mesh Regular/1″×4″/1.8 g./1.3/1.39 times test/24+liter/Comfortable/Acceptable

3.Mesh Light/0.75″×4″/1.3 g./1.3/same as test/17+ liter/V. Comfortable/Very Comfortable

4.Mesh Mini/½″×3.5″/0.8 g./1.3/62% of test/10+liter/Not Noticeable/Not Tested

5.Mesh Mini/2.75″×3″/1.0 g./1.3/77% of test/13+liter/Not Noticeable/Extr. Comfortable

6.Extruded Reg./(¼″tk) 1″(4.5″)×4″/7.5 g./1.75/4.29 times test/74+liter/Comfortable/Not Tested

7.Extrud. Mini2/(⅜″tk)0.75″(4.5″)×3″/3.1 g./1.75/1.77 times test/30+liter/Comfortable/Not Tested

8. Mesh Comfort/1.5″×3.5″/2.0 g./1.3/1.54 times test/26+liter/Comfortable/Comfortable

Results of Testing Variations of the Preferred Embodiment—

[1] The simple basic rectangular shape, with the flexibility and compressibility inherent in these filtering media materials were able to conform to the contours of the body to achieve at least an acceptable level of comfort in almost all sizes and test conditions.

[2] Only the Mesh Maxi size in the 8 hour sitting test (simulating conventional office work with ˜90% of the time seated) was uncomfortable. A briefer sitting test of about 3 to 4 hours duration did achieve an acceptable comfort level.

[3] In most cases, “discomfort” was the feeling of pressure under the Coccyx. Relieving this pressure achieved comfort, and was accomplished by reducing the width and/or shortening the length of the matrix material. Once the wearer becomes accustomed to the presence of the filter (after about two uses), the presence of the filter, in and of itself, did not cause discomfort.

[4] Concerning effectiveness of the various filters, substantial testing has already been performed. Assuming both output ends are equally used, we can theoretically expect the same performance from the filters on a per media-ounce basis, as were observed in the odor absorption lab tests. Both media types were lab tested to 17.4 liters of gas cleansed, so modification #3 (see table above), holding the same gram weight of media, should cleanse the same volume of gas, and modifications #4 and #5, with less weight of media, would both still cleanse less than 17.4 liters, but more than 10 liters of gas. The four other modifications all use more than the researched lab weight of media, so these will cleanse proportionally more than 17.4 liters of gas, as shown above. The smallest projected volume of cleansed gas (10+ liters) is several times more than most people can be expected to need in an entire day. In practice, all these tested modifications (i.e., modifications #1-8 below) should provide more than enough active ingredient than will be needed in the maximum life of the product, which would be a full workday. It may be desirable to increase the expected filtering capacity in order to accommodate various arbitrary factors, such as shelf life deterioration, manufacturing inconsistencies, user contamination of the product, user blocking of one outlet completely (which cuts the capability in half), etc. Even so, the filtering medium will not require any more carbon than the smallest amount tested. An exception to this may be the shortest media lengths. Very short media length, with half the media from the saddle to the outlet end, may not adequately reflect the carbon density of the lab tests, and some odor might escape or bypass very short lengths of mesh media.

Returning now to FIG. 5, different matrix material versions of the Safe-T Thong embodiment are shown in FIGS. 5(a)-5(f). At FIG. 5(a), we see the Mesh Maxi filter version, which is a 6″×1″ media rectangle with both ends symmetrical with respect to the hole 43 location. This design has, at present, been tested most extensively. This large configuration can be shortened by one inch (dotted line on FIG. 5(a)), to give more room at the back of the filter, under the coccyx, for much greater comfort in seated positions, making the geometry of the filter non-symmetrical with relation to the hole 43. In cases where the filter does not have bi-symmetry, an indication may be provided to the user for which end of the filter goes to the “front” (such as the dotted arrow shown). In FIG. 5(b) we see the Mesh or extruded Regular embodiment (shortened to 4″). Shortening the filtering media simplifies the unit by making it symmetrical, and it consequently has no specified “front” end. While the shorter length will not last as long as the larger version, the research above demonstrates that the smaller size units provide more than adequate performance due to the high absorptive capability of these media material types.

A “high comfort”, or “athletic version”, is a precut, ergonomically contoured media shape which matches the human body's shape, as shown in FIG. 5(c). This shape requires a “front” end indicator (dotted arrow), and strenuous athletic activity will require the maximum comfort, and be worth the increased cost required to build a more complex shape. However, simply wearing any filter causes media compaction (or rearrangement in the case of an extruded filter), which quickly reduces the standard rectangular shape to custom ergonomic three-dimensional contours. The precut benefit may not be worth the added cost, to construct an ergonomically shaped filter.

The filter design shown in FIG. 5(d) is conceived as a “contoured comfort” version, and is essentially ergonomic, but has been made symmetrical for the sake of simplicity. This embodiment is comfortable, even for sleeping.

An extruded media embodiment of a filter is shown in FIG. 5(e). The preferred way to use this material to form a filter is as follows. First, the top scrim 51 is peeled off and discarded. The particulate carbon 52, still attached to the bottom scrim, is rolled (i.e., rolled scrim-side “in”), into a 1″ high by six layers thick shape 53. The barrier film 41 and fabric 42 are then wrapped around the consolidated form 53 as previously described, with the hole 43 in the barrier film disposed at top center. This configuration is equivalent to the 5B design illustrated in FIG. 5(b) for a generic filtering medium. The Mesh Mini, the smallest embodiment of a filter built to date by the present inventor, is illustrated in FIG. 5(f).

Alternative Filter Embodiments

There are many alternative configurations which can be constructed using the fundamental concepts set forth herein. Examples of such alternative embodiments include the Fabric Bag, the Perforated Sleeve, and the Pancake embodiments as will be discussed below.

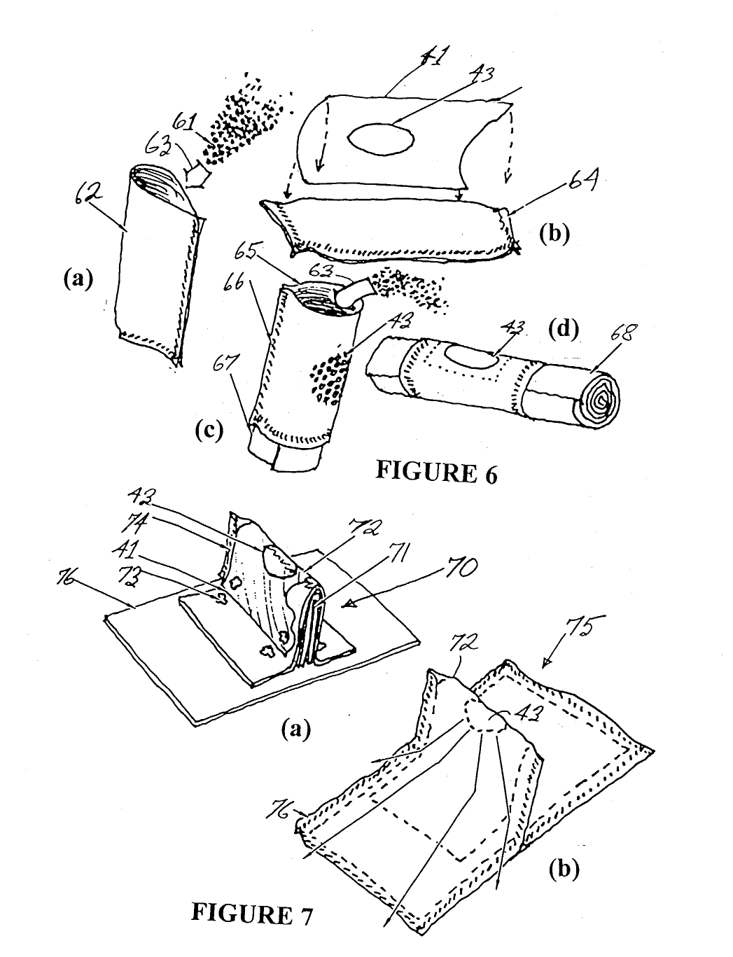

The Fabric Bag configurations, shown in FIGS. 6(a)-(d) use a loose carbon particulate 61 as a filtering medium. A bag 62 may be formed by heat sealing a non-woven fabric 62, and filling with carbon 61 as indicated at broad arrow 63. Either mixtures or single forms of granular, pelletized, powder, or beaded carbon 61 can be used. Heat sealing the end openings of the (carbon-filled) bag produces a completely sealed package of carbon 64. A barrier film material 41 having a hole 43 therein is then wrapped around the carbon-filled bag 64 (dotted arrows). These steps provide a sub-assembly that is equivalent to the embodiment shown in FIG. 4(b), which may now be wrapped with fabric and finished according to instructions for FIG. 4, to form a filter employing any particulate, chopped, or loose form of activated carbon.

A Perforated Sleeve embodiment of a filter in accordance with the present invention is shown at FIG. 6(c) and may be a continuous sleeve material 65 such as a tubing, or formed from a film, by heat sealing along a seam 66. A non-woven fabric 67 can be incorporated into the ends by heat sealing, or a single thick plug or roll of “breather” material may be heat sealed into the end before filling the sleeve 65 with a particulate carbon as at broad arrow 63. The breathable saddle (hole 43) which permits gas entry into the filter may be formed by punching a plurality of small holes in the fabric, as shown in FIG. 6(c), or a non-woven liner can be heat sealed over an open hole 43 as illustrated in FIG. 4(d), to cover the opening. After filling, if a fabric is used, the sleeve can be heat sealed, or an extension 68 of a porous breather material can be provided for greater comfort under the coccyx.

A Pancake embodiment of the filter is illustrated at numeral 70 in FIG. 7. The filter embodiment shown at 70 in FIG. 7(a) uses sheets of extruded carbon 71 to form a filter insert section 72 that fits into the crevice between the buttocks adjacent the anus. The sheets of extruded carbon are connected to one another by stitching, bonding, or hot melt “spot weld” 73 to form a flat, thin “pancake” of extruded filter material. The crevice insert section 72 is covered with a barrier film 41, which is heat sealed at front (front seal not shown in FIG. 7(a)) and back edges as indicated at 74, and has a conventional hole 43 cut therein to permit gas entry into the filter. The subassembly is heat sealed within a fabric envelope as illustrated in FIG. 7(b) to provide a filtering device 75. Most of the mass and bulk are at the insert section 72, which is placed in the crevice in the same manner as the thong-type filter configuration. This variation is a “multiple matrix equivalent”, since gas may take many routes through and out of the filter 75 (such as arrows emanating from hole 43), and all routes are equivalent to a linear matrix filter. All gas must exit through the pancake layer 76.

“Thong” Method of Filter Configuration and Placement

Many problems associated with filtering odor from human gas derive from difficulties associated with that particular location of the body, which challenge a filter to be both technically effective, and practical for normal everyday use. The present invention proposes a preferred novel filter configuration and method for filter placement that is vaguely reminiscent of “Thong” underwear. Basically, for the purposes of odor filtration, if activated carbon is placed in direct contact with the gas exit location of the human body, the body crevice adjacent to that location may be utilized for placement of the filter. Details of the device must be very specific for this filter location to be acceptable technically, and to be practical in use. Research for the present invention has shown that with the correct designs and materials, this method is extremely effective. The exact filtration process and filtration methods and materials were discussed above, but the Thong method of placement and configuration will be described here.

The crevice between the buttocks adjacent the anus is dimensioned to receive and provide concealment for the filtering devices of the present invention. Most filter embodiments of the present invention can easily be at least partially concealed, and some can even be fully concealed within this crevice. Worn under clothing, concealment of all embodiments of the filter is possible. Having established that this crevice is an ideal location for a gas filter, there remains the requirement to make the concealed filter technically effective, comfortable, and easily secured in place.

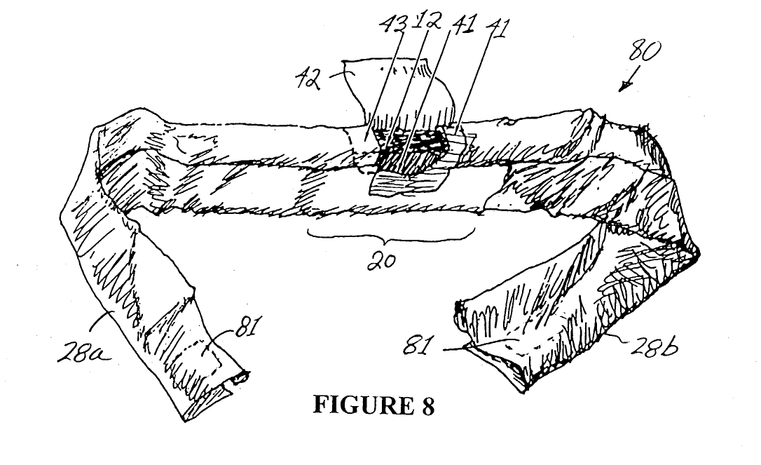

In order to be effective, the filter must provide means for overcoming gas blocking, should it occur, and route gas quickly through the filter, stripping all odor from the gas stream during transit through the filter. As shown above, extensive testing for the present invention substantiates all materials performance requirements. It is apparent that gas immediately enters the filter as it exits the body because the filter input location is directly against the body's gas exit location as illustrated in FIG. 2. With alternate reference to FIGS. 2 and 8, a thong filter is shown in FIG. 8 at numeral 80. The Thong filter 80 is in direct contact with the body along the entire crevice (FIG. 2), and the body's gas exit location 23 is in contact and aligned with the gas input location 24 of the filter 20. All tested fabric materials meet the requirement that the filter's gas input location readily admit gas at a high permeability rate, and provide a very thin and porous covering over the filter media. All tested barrier films demonstrate required gas flow control. All tested filtering media provide satifactory, unobstructed gas flow through the filter, even while compacted, and the best performing media materials provide more than enough odor stripping capability, even in reduced media-volume embodiments.

Filter Comfort—The lab tests and media configuration comparisons demonstrate that the highest performing active ingredients will easily permit some reduction of the size of the filter without sacrificing essential filtering performance, thereby enabling the filtering device to fit comfortably within the crevice. Of particular importance is comfort during seated positions of the user. In seated positions, the coccyx is close to, and in some cases in direct contact with, the seat surface. Any objects located between the seat surface and the coccyx can become a source of discomfort. Consequently, the most comfortable filter designs of the present invention have a smaller cross-section for reduced bulk under the coccyx when in a seated position, or the filter length is sufficiently short that it presents no bulk under the coccyx when the wearer is in a seated position.

Ergonomic aspects play a large part in the comfort experienced by the filter user. The linear shape of the receiving body crevice is exploited by making a linear filter having a slim profile. If the complete filter is also flexible, it curves comfortably into the crevice. Since the filter is compliant, we have the ability for the filter to be reshaped during use to assume a “custom ergonomic” shape under natural compaction forces which take place in the crevice. All these compacting conditions exist with all filter embodiments, even with extruded media. Through the natural body movements of sitting, standing, walking, running, etc., each filter becomes somewhat compacted and reshaped for a custom fit within the particular crevice. Test filters built like this are so comfortable that test subjects forget they are wearing a filter, and can easily wear one for 10 hours or more in comfort.

Novel Secure Attachment

To eliminate the problematic and ineffective attachment devices employed with prior art devices, the present invention presents a completely different method for attaching a filter to the body: surface area contact attachment. The present method employs several structural and functional improvements which, taken together, cause the filter to stay securely adhered to the skin through friction alone, so that no other form of filter attachment is needed to keep it in place. These improvements are:

1. Correct position—the filter saddle (i.e., the portion of the filter comprising the hole in the barrier film) is centered on the body gas exit location, and directly against the gas exit, with the filter positioned all the way into that body crevice along the entire length of the filter.

2. Enlarged surface area—when the filter is in place, the outer surface of the filter portion, which filter portion houses the activated carbon, constitutes most of the circumferential surface area of the housing. The outer surface area of the filter can only increase with compaction, thus increasing adhesion. This increase in adhesion of the filter to the body due to increasing outer surface area under compaction counteracts the slight loss in adhesion caused by lower pressure against the outer surface due to compaction of the filter. The net change seems to be an overall increase in adhesion as the filter undergoes compaction, but there may be other phenomena at work that contribute to this overall adhesion increase during compaction.

3. Non-slippery fabric—the outermost fabric covering of the filter is preferably made of a material which tends to cling to the skin if pressed against the skin with only slight pressure. This requirement can be met in several ways. Some materials automatically possess adhesive sharacteristics, such as nonwoven Mexican polyester which was tested and found to be the best in this respect of the several fabrics that were tested. Some materials can be modified to provide a nonslip surface, such as nonwoven trilobal nylon. Some materials can be modified by applying a non-sticky adhesive layer to the outer surface of the filter, such as a brush-on or spray-on rubber cement type of material, or assisted by a pressure sensitive transfer adhesive on the straps can be applied to the user's underwear.

4. Pressure must be applied to both sides of the filter, a condition which is automatically met when the filter is placed in the correct position within the crevice, where the sides of the crevice are always in constant pressure against the sides of the filter. All correctly positioned filters that were constructed with nonwoven Mexican polyester achieved secure and non-slipping attachment to the body within the crevice. Some materials required adhesive patches to attach the straps to the underwear.

Safe-T Filter Placement—correct placement of the Safe-T Thong filter is required for proper operation. The user takes the filter in one hand, holding the filter at the center between thumb and fingers, just below the outlined gas input manifold (the outline comprising a visual indicia printed on the outer surface of the filter that resembles a saddle, indicating the position of the hole in the underlying barrier film). In a bent-over standing position, the user places the manifold center (or saddle) directly against the body's gas exit location. If the saddle is in direct body contact, gas will exit the body with little or no sound. Holding the manifold in place, the user stands completely straight, tucking the filter 20 snugly into the body's crevice all along the length of the filter, and gently pulls both straps 28a and 28b straight up, and then replaces clothing. If the filter becomes dislodged, as, for example, through activity, the straps can be used to pull the ends of the filter back into position without removing clothing. If there is any concern about straps showing above or through certain clothing styles, the straps can be cut shorter, or even cut off completely. The straps 28a and 28b, which preferably comprise adhesive means 81 operable for releasably attaching the front strap 28a and the rear strap 28b to the top edge of the undergarment, should not be cut off completely unless necessary, as they help guide the clean filtered gas out from the body. The filter device 80 will fit the contour of the body underneath an undergarment in a manner similar to thong underwear, but will feel much more comfortable than thong underwear. If the user has never worn thong underwear, wearing a filter will feel strange initially, but not uncomfortable-especially when first used. After the second or third day of wearing a filter, it will be no more noticeable than wearing any other article of clothing. Used filters should be disposed of in the trash, not flushed down the toilet.

Method of Eliminating Gas Sound

The only example of prior art which attempts to overcome the sound of gas release is Grosse, who may have attenuated the sound by muffling it with his thick absorbent pad structure. The present invention uses a completely different method. This new method literally prevents generation of the sound at the point of the sound's origin, and does this without interfering with actual gas flow.

The sound accompanying gas release is due to vibration of the gas exit orifice while gas is exiting the body. When a stationary material comes in contact with a resonant vibrating mass caused by passage of air, vibration is damped and stops, even while the gas release which initially caused the vibration continues unabated. This same principle is applied by the present invention. In FIG. 8, a cut away view of the high permeability fabric 42, reveals the exposed media 12 within the saddle 43, which saddle is a hole cut in the barrier film layer 41. The exposed patch of media within the saddle, lies beneath and directly contacts the underside of the fabric. When the filter is put in place, this high air permeability fabric 42, is placed in direct contact with the body's gas exit orifice. Sometimes the underlying bagged carbon media 12 is also involved in the fabric contact with the gas exit orifice. When gas is exiting the body, this direct contact with the fabric is sufficient to prevent vibration of that orifice; and thus, no characteristic gas noise is generated. It takes only slight contact with this fabric to prevent vibration of the orifice, and with correct placement of the filter, the contact is usually retained all the time, whenever a filter is used. Consequently, the sound accompanying escaping gas is reduced or prevented by the same high air permeability fabric material that permits gas to enter the filter through the saddle.

Method of Assuring Gas Flow

Filter bypass was one of the four reasons for the failure of prior art filter devices (see Prior Art above), where thick bulk, intended to absorb gas, in fact constituted a formidable barrier to gas penetration. This is especially true in instances of abrupt release of a large volume of gas. Subject testing of the Thong Method of body gas odor filtration has verified that the method assures that gas flows through the filter, rather than around it. Referring to FIGS. 2 and 3, the method of employing a thong-supported filter to filter odor from gas released by the body consists of the following parts:

1. Direct contact of the body gas exit location 23 with the gas input location 24 of the filter 20, with the linear filter 20 positioned all the way into the body gas exit location crevice all along the length of the filter. This filter position does two things: (a) it provides immediate body access to a low resistance, high flow rate route for gas to take as it exits the body; and (b) compaction forces within the crevice provide a reasonably good seal all around the gas intake manifold 24 (saddle), which prevents the gas from finding alternate routes to by-pass the filter.

2. A high air permeability filter-covering fabric 28 is directly in contact with the body gas exit location 23, and covering 28 is the only barrier which gas entering the filter 20 must pass through before entering the activated carbon odor filtration media 12.

3. The filter employs an activated carbon media composition which maintains good air permeability characteristics that enables a gas to flow through the carbon (FIG. 2, broad arrows 13a and 13b), even while subject to compaction forces experienced by the filter within the crevice of the body gas exit location (all tested carbon media exhibited this characteristic).

4. The method uses a filtering device enabling cleansed gas to easily flow away from the filter (both front and back ends) after exiting the carbon filter portion, either along the folds of the fabric straps or directly through the strap material (FIG. 3, arrow 14), which strap 28 is a continuous piece of the same high air permeability material covering the filter's gas entry location.

These characteristics of the filter, taken together, when placed in contact with the body and supported against the gas exit orifice of the body as discussed hereinabove, provides a method for directing gas flow through the filter and preventing filter bypass, and provides means by which exiting body gas finds the most favorable route (the path of least resistance) away from the body, which route will necessarily pass through the filter.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What I claim is:

1. An odor filtering device operable for removing odiferous molecules from a gas stream exiting a body through an orifice in the body, the device comprising: (a) an elongate filter comprising activated carbon, said filter having open ends providing a plurality of gas exit ports and a body portion therebetween, a centrally located portion of said body portion comprising a gas input port operable for receiving the gas stream; (b) a barrier film that is substantially impermeable to the gas, said barrier film enveloping said body portion of said filter and having a hole therein wherein said hole is disposed to overlie said gas input port on said body portion of said filter; and (c) a gas permeable fabric overlying said barrier film, said open ends of said filter and said hole in said barrier film, wherein said filter, said barrier film and said fabric form an elongate odor filtering device having integral construction.

2. The odor filtering device in accordance with claim 1 wherein said fabric extends beyond said ends of said filter, said portion of said fabric extending beyond said ends of said filter defining straps operable for supporting said hole in said barrier film and said gas input port adjacent the orifice in the body.

3. A method for removing odor from human body gas comprising the steps of (a) presenting an odor filtering device in accordance with claim 1; then (b) placing the hole in the barrier film adjacent the orifice on the body.

4. A method for removing odor from human body gas comprising the steps of (a) presenting an odor filtering device in accordance with claim 2; then (b) placing the hole in the barrier film adjacent the orifice on the body; then (c) donning an undergarment to overlie said odor filtering device; then (d) attaching said straps to said undergarment to securely maintain the position of the hole in the barrier film adjacent the orifice.

5. The odor filtering device in accordance with claim 1 wherein said fabric adheres to a portion of skin on the body adjacent to the orifice through which the stream of gas exits the body.

6. A method for removing odor from human body gas comprising the steps of (a) presenting an odor filtering device in accordance with claim 5; then (b) placing the hole in the barrier film comprising the filtering device adjacent the orifice on the body; the adhesion of the fabric to the skin thereafter being sufficient to support the filtering device such that the hole in the barrier film remains adjacent to the orifice.
